(19) United States
(12) Patent Application Publication
(10) Pub. No.: US 2022/0169593 A1
(43) Pub. Date: Jun. 2, 2022

(54) Alkyl-Aryl Amine-Rich Small Molecules and Their Composites with Solid Mesoporous Substrates

(71) Applicants: Georgia Tech Research Corporation, Atlanta, GA (US); Global Thermostat, New York, NY (US)

(72) Inventors: Christopher W. Jones, Atlanta, GA (US); Dharam Raj Kumar, Atlanta, GA (US); Eric W. Ping, New York, NY (US); Cornelia Rosu, Atlanta, GA (US); Miles A. Sakwa-Novak, New York, NY (US); Achintya Sujan, Atlanta, GA (US)

(21) Appl. No.: 17/338,060

(22) Filed: Jun. 3, 2021

Related U.S. Application Data
(60) Provisional application No. 63/034,001, filed on Jun. 3, 2020.

(51) Int. Cl. C07C 209/06; B01J 20/10; B01J 20/28; B01J 20/32

(52) U.S. Cl. CPC C07C 209/06; B01J 20/103; B01J 20/28073; B01J 20/3248; B01J 20/28061; B01J 20/3204; B01J 20/28059

(57) ABSTRACT

The present disclosure provides for alkyl-aryl amine-rich small molecules that are prepared by nucleophilic substitution from tri- and hexa-bromine-substituted aromatic cores with various aliphatic diamines. The resulting products can be subsequently subjected by solution impregnation into solid mesoporous supports. Various types of alkyl-aryl amine-rich small molecules can fill the support's pores up to ˜90% and displayed good thermal stability

### CROSS REFERENCE

This application claims priority to, and the benefit of the contents of U.S. provisional application entitled “Alkyl-Aryl Amine-Rich Small Molecules and Their Composites with Solid Mesoporous Substrates” having Ser. No. 63/034,001 filed on Jun. 3, 2020, which is entirely incorporated herein by reference.

### ABSTRACT

Alkyl-aryl amine-rich small molecules (Ph-XX—YY) were prepared by nucleophilic substitution from tri- and hexa-bromine-substituted aromatic cores with various aliphatic diamines. The resulting products were subsequently subjected by solution impregnation into solid mesoporous supports. Various types of Ph-XX—YY molecules filled the support's pores up to ˜90% and displayed good thermal stability.

### SUMMARY

The present invention discloses a versatile one-pot synthetic method to prepare different types of amine-rich small molecules (Ph-XX—YY) of various compositions. Nucleophilic substitution was employed to react tri- and hexa-bromine-substituted aromatic cores with excess ethylenediamine and propane-1,3-diamine. The excess diamine prevents oligomerization and favored high yields of products. Synthesis of alky-aryl amines was confirmed by ¹H, ¹³C NMR and high-resolution mass spectrometry (HR-MS). Also described composite materials made of various organic loadings of Ph-XX—YY molecules combined with various mesoporous support materials; first with SBA-15 mesoporous silica a prototypical example, through solution impregnation. In addition, porous supports such as alumina, carbon, titania, porous polymers, and MOFs can also be used. The wet impregnation approach yielded composite materials, such as Ph-XX—YY/SBA-15. Thermogravimetric analysis (TGA) and differential scanning calorimetry (DSC) were used to investigate the thermal behavior of the pure Ph-XX—YY and composite materials. It was found that encapsulation in the SBA-15 pores shields Ph-XX—YY molecules, slowing their thermal degradation. N₂ physisorption analysis was used to assess the porosity of the composite materials and determine the pore structure parameters of the Ph-XX—YY/SBA-15 composites. The specific BET surface area decreased with the increase in Ph-XX—YY loadings, a trend also followed by the total pore volume and average pore size. These results confirmed the presence of Ph-XX—YY within the cylindrical pores of SBA-15. Together the data indicate that, due to their good thermal stability and uniform distribution within mesoporous solid supports, Ph-XX—YY/solid mesoporous support (e.g. silica, γ-Al₂O₃, porous carbon) composites are appealing platforms for various applications, in particular integration into direct air CO₂ capture (DAC) or other CO₂ capture technologies as well as H₂S capture.

### BACKGROUND

Benzene substituted with various functional groups has been found as an appealing core for the preparation of star-like small molecules and polymers. Sun et.al prepared a series of polymers by facile nucleophilic substitution reactions using inexpensive and readily available 2,4,6-tris(chloromethyl)mesitylene (TCM) and diamine monomers.^{1, 2} Because of the halogen group's affinity to amines, the reaction was carried out at a mild temperature (60° C.) and did not require any catalyst. Two invention disclosures by Looper et al. have described the methods for preparing a variety of polyamines containing an aromatic core anchored by one, two and three amine-rich side arms.^{3, 4} In order to achieve the desired biocidal activity, the authors have used a three-step synthesis that, in the case of some products, required protection of one terminal primary amine of spermine and of its polyamine homologues. In addition, the preparation also required the use of metal catalysts (e.g. Pd) and pyrophorics (e.g. NaH).³ In another multi-step approach. Stawicki et al. have reported dipodal and tripodal amine scaffolds that were subsequently used to prepare ligand adducts for in-vivo uranyl removal. In order to prepare these amines, the authors have reacted bis- and tris-bromo-methyl-aryl derivatives with excess diamine (e.g. ethylenediamine, 1,3-propylenediamine, 1,4-butandiamine).⁵ A relatively similar route was proposed by Guo et al., but, additionally, they have explored 1,3,5-tricarboxyaldehides to synthesize polyamino tripodal ligands.⁶ The diamines used, N-ethyl-ethylenediamine, N-n-propyl-ethylenediamine and N-n-butyl-ethylenediamine were more complex and richer in primary and secondary amine groups than those reported by Stawicki et al.⁶⁻⁸ The same tricarboxyaldehydes were used by Still et al. in an invention that synthesized a series of polyamine molecules for use as synthetic receptors.⁹ The composition of amine-containing materials described in these examples was highly complex because their target applications required multifunctionality. Reaction of aromatic dialdehydes and guanidine groups recently reported by Seipp et al. has yielded crystalline, amine-rich molecules for use as adsorbents in CO₂ capture.¹⁰ In their approach the authors have used an aqueous solution of the synthesized 2,6-pyridine-bis(iminoguanidine) that was allowed to slowly crystallize in ambient air conditions. CO₂ and water were adsorbed as a part of a crystal hydrate form that, upon heating at 120° C. were released, according to optical microscopy (crystal color change) and thermogravimetry. Indeed, this is another example of innovative approach but it is rather challenging to achieve good CO₂ adsorption/desorption properties. The departure of CO₂ from the crystalline phase requires elevated temperature that makes the process costly.

Aromatic carboxylic acids were also investigated for their reactivity toward amines as a potential pathway leading to amine-rich compounds. Nagata et al. have reported aliphatic-aromatic and aromatic polyimines through reaction of trimesic and pyromellitic acids with hexa-, deca-, and dodecamethylenediamine.¹¹ Melt condensation between trimesic acid and diamines (e.g. hexamethylene, dodecamethylenediamine) was employed by Kiyotsukuri et al. to prepare transparent polyamide films that were insoluble in organic solvents.¹² Additionally, the authors have used aromatic diamines such as 4,4′-oxydianiline, 1,4-phenylenedimethanamine, benzene-1,4-diamine, benzene-1,3-diamine and piperazine that reacted with trimesic acid afforded aromatic polyamido-amines with high resistance to thermal degradation.¹³ Similar hyperbranched polyamides derived from trimesic acid were also reported by Jikei et al.¹⁴ Edwards and Robinson have disclosed the synthesis of organic salts, namely poly(pyromellitimide)s.¹⁵ In order to afford high-molecular-weight salts, the authors converted pyromellitic acid to its aldehyde homologue that was subsequently reacted with 4,4-dimethylheptamethylene diamine, nonamethylene diamine and 3-methylheptamethylene diamine. Recently, pyromellitic dianhydride was used in combination with Jeffamine D-400 by Yu et al. to prepare amine hardeners.¹⁶ Investigations performed by Kumar et al. showed that the reaction mechanism between pyromellitic dianhydride and aromatic diamines occurred in two stages.¹⁷ First the formation of a prepolymer complex was observed, which in the presence of excess dianhydride led to high molecular weight poly(amic acid).¹⁷ While these polyamido-amines presented an increase in resistance to thermal degradation, their synthetic route may be regarded as a difficult to scale up process due to the associated costs, especially for technologies such as DAC.

Other amine-rich molecules used in tandem with solid supports have been explored as adsorbent materials for DAC as well as other mixtures of gases, such as natural gas and flue gases.¹⁸ The most used method for preparation of these composite materials is solution impregnation leading to both physical binding of amine molecules to the substrate surface and partial or complete pore filling.^{19, 20} Depending on their porosity,²¹ solid supports are able to carry varied amounts of amine content.²² They also prolong the life time use of amine active sorbents because of their improved stability under ambient and thermally treated conditions. In this regard, mesoporous silicas,^{19, 23-25} alumina,^{26, 27} zeolites,²⁸ graphene oxide,²⁹ porous polymers,³⁰ carbons,^{31, 32} MOFs^{33, 34} and carbon nanotubes^{35, 36} are a few examples of supports investigated for extending the library of amine-containing composite materials for CO₂ capture.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIGS. 1A-1D: TGA curves for (FIG. 1A) Ph-3-ED/SBA-15, (FIG. 1B) Ph-3-PD/SBA15, (FIG. 1C) Ph-6-ED/SBA-15 and (FIG. 1D) Ph-6-PD/SBA-15 composites. Each graph contains the trace of pure Ph-X—YY molecule.

FIGS. 2A-2D. Differential scanning calorimetry profiles of (FIG. 2A) Ph-3-ED/SBA-15, (FIG. 2B) Ph-3-PD/SBA-15, (FIG. 2C) Ph-6-ED/SBA-15 and (FIG. 2D) Ph-6-PD/SBA-15 composites. Each graph contains the trace of pure Ph-XX—YY.

FIGS. 3A-3D: Nitrogen physisorption isotherms for (FIG. 3A) Ph-3-ED/SBA-15, (FIG. 3B) Ph-3-PD/SBA-15, (FIG. 3C) Ph-6-ED/SBA-15, (FIG. 3D) Ph-6-PD/SBA-15 composites of various organic loadings (0-50%) recorded at 77K.

FIGS. 4A-4D Pore size distribution for (FIG. 4A) Ph-3-ED/SBA-15, (FIG. 4B) Ph-3-PD/SBA-15, (FIG. 4C) Ph-6-ED/SBA-15 and (FIG. 4D) Ph-6-PD/SBA15 composites. Each graph contains the trace of neat SBA15.

FIGS. 5A-5B. (FIG. 5A) TGA and (FIG. 5B) DSC traces of Ph-XX—YY/mesoporous carbon and Ph-XX—YY/γ-Al₂O₃.

FIGS. 6A and 6B. Nitrogen physisorption isotherms for (FIG. 6A) Ph-3-ED/mesoporous carbon and (FIG. 6B) Ph-3-ED/γ-Al₂O₃ composites of 0% and 50% organic loading recorded at 77K.

FIGS. 7A and 7B. Pore size distribution for (FIG. 7A) 50% Ph-3-ED/CNP and (FIG. 7B) 50% Ph-3-PD/γ-Al₂O₃ composites. Each graph also contains the trace of the neat substrate CNP and γ-Al₂O₃.

FIGS. 8A-8H illustrates NMR images of various compounds.

### DESCRIPTION

### Reagents

TABLE 1
Bromine-Substituted Aryl Cores and Diamines Used for the
Synthesis of Aryl-Alkyl Amine-Rich Molecules (Ph-XX-YY). Where,
Ph— stands for phenyl moiety, XX stands for number of substitutions
on phenyl ring (3 or 6) and YY stands for ED (ethylene diamine) or
PD (propane- 1,3-dia mine).

| Bromine-substituted | |
|---|---|
| aryl cores | Diamines |
| 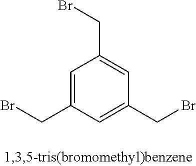 [SMILES: BrCc1cc(CBr)cc(CBr)c1] | 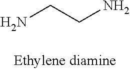 |
| 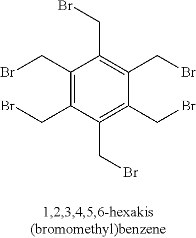 [SMILES: BrCc1c(CBr)c(CBr)c(CBr)c(CBr)c1CBr] | 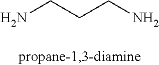 |

Ph-XX-YY

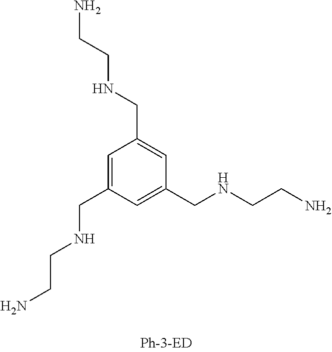 [SMILES: NCCNCc1cc(CNCCN)cc(CNCCN)c1]

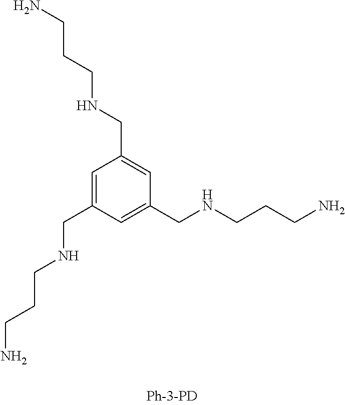 [SMILES: NCCCNCc1cc(CNCCCN)cc(CNCCCN)c1]

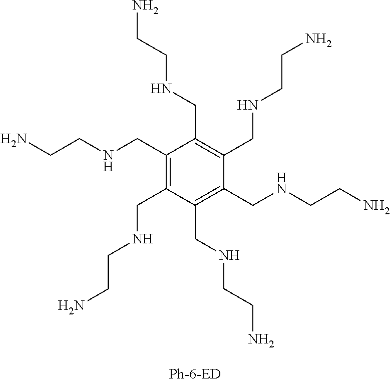 [SMILES: NCCNCc1c(CNCCN)c(CNCCN)c(CNCCN)c(CNCCN)c1CNCCN]

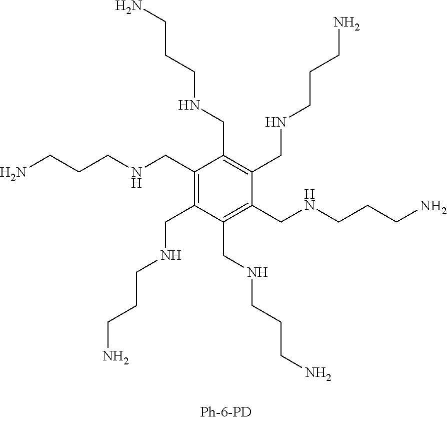 [SMILES: NCCCNCc1c(CNCCCN)c(CNCCCN)c(CNCCCN)c(CNCCCN)c1CNCCCN]

### EXPERIMENTAL

### 1. Reaction of 1,3,5-tris(bromomethyl)benzene (Ph-3-CH₂Br) with ethylene diamine (ED)

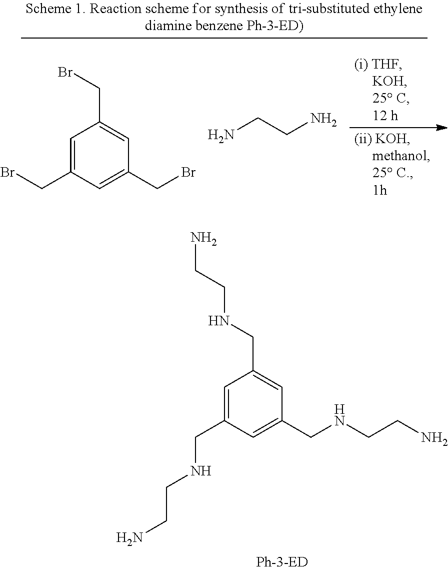

### EXPERIMENTAL

### 2. Reaction of 1,3,5-tris(bromomethyl)benzene (Ph-3-CH₂Br) with ethylene diamine (ED)

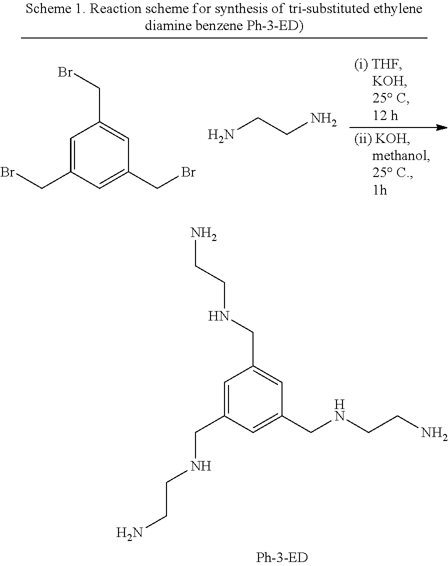

Synthesis of Ph-3-ED. 1,3,5-tris(bromomethyl)benzene (2 g, 5.6 mmol) was dissolved in dry THF (60 mL) under argon at room temperature. This solution was added dropwise to excess ethylenediamine (13.5 g, 224 mmol) at room temperature under argon. After complete addition, the reaction mixture was stirred overnight at room temperature. The solvent and excess ethylenediamine were removed under reduced pressure (rotary evaporation). Subsequently, the resulting brown viscous oil was dissolved in methanol (15 mL). Potassium hydroxide (0.9 g, 16.8 mmol) was added and the mixture was stirred for 1 hour. The inorganic salt (KBr) was precipitated by addition of diethyl ether and removed by filtration. The filtrate was rotary evaporated to afford a high viscous yellow oil (1.58 g, 96% yield). ¹H NMR, (D₂O, 700 MHz), δ (ppm): 7.21 (3H, s), 3.73 (6H,s), 2.71 (6H,t), 2.61 (6H,t). ¹³C NMR, (D₂O, 175 MHz), δ (ppm): 139.76, 127.39, 52.14, 50.01, 39.82. m/z: found; [M+H]⁺ 295.2607, calc. for [M+H]⁺ 295.2605.

### 2. Reaction of 1,3,5-tris(bromomethyl)benzene (Ph-3-CH²Br) with propane-1,3-diamine (PD)

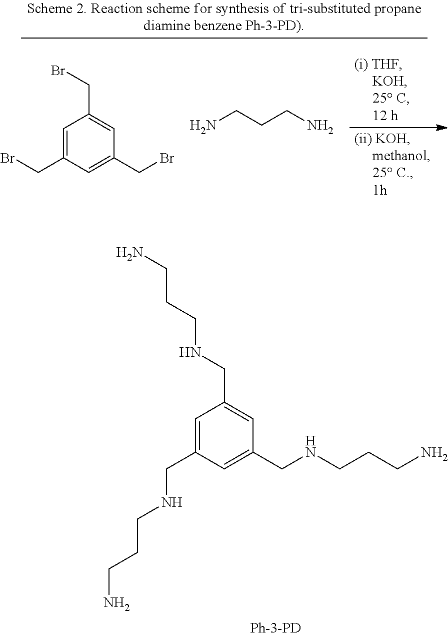 [SMILES: BrCc1cc(CBr)cc(CBr)c1 ; NCCCN ; NCCCNCc1cc(CNCCCN)cc(CNCCCN)c1]

Synthesis of Ph-3-PD: 1,3,5-tris(bromomethyl)benzene(2 g, 5.6 mmol) was dissolved in dry THF (60 mL) under argon at room temperature. This solution was added dropwise to excess propane-1,3-diamine (16.6 g, 224 mmol) at room temperature under argon. After complete addition, the reaction mixture was stirred overnight at room temperature. The solvent and excess propane-1,3-diamine were removed under reduced pressure. Subsequently, the resulting colorless viscous oil was dissolved in methanol (15 mL). Potassium hydroxide (0.95 g, 16.8 mmol) was added and stirred for 1 hour. The inorganic salt (KBr) was precipitated by addition of diethyl ether and removed by filtration. The filtrate was rotary evaporated to afford a highly viscous yellow product (1.8 g, 95% yield). ¹H NMR, (D₂O, 700 MHz), δ (ppm): 7.20 (3H, s), 3.72 (6H, s), 2.67 (6H, t), 2.58 (6H,t), 1.65 (6H, q). ¹³C NMR, (D₂O, 175 MHz), δ (ppm): 139.56, 127.52, 52.17, 45.38, 38.48, 30.48. m/z: found; [M+H]⁺ 337.3078, calc. for [M+H]⁺ 337.3074.

### 3. Reaction of 1,2,3,4,5,6-hexakis(bromomethyl)benzene (Ph-6-CH2Br) with ethylene diamine (ED)

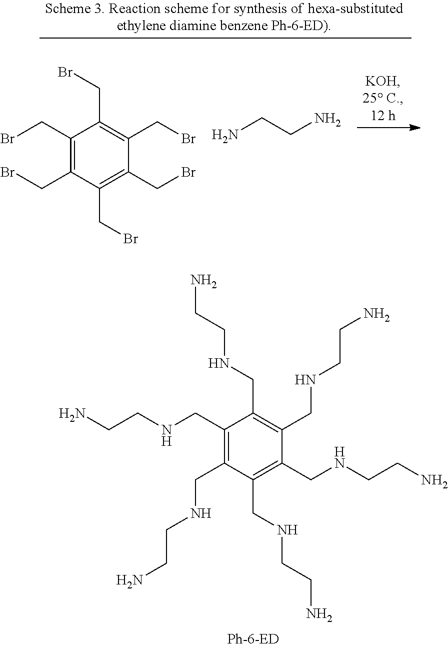

Synthesis of Ph-6-ED: In a round bottom flask 1,2,3,4,5,6-hexakis(bromomethyl)benzene (1.5 g, 2.36 mmol), excess ethylenediamine (8.5 g, 141.5 mmol) and KOH (0.7 g, 14.2 mmol) were added under argon atmosphere. The reaction mixture was stirred overnight at room temperature. Excess diamine was removed under reduced pressure (rotary evaporation). The resulting brown and viscous product was dissolved in methanol (10 mL). The inorganic salt (KBr) was precipitated by addition of diethyl ether and removed by filtration. The filtrate was rotary evaporated to afford a highly viscous brown liquid. The product was dried on a high vacuum line to remove trace amine (0.7 g, 58% yield). ¹H NMR, (D₂O, 700 MHz), δ (ppm): 3.80 (12H, s), 2.80 (24H, m). ¹³C NMR (D₂O, 175 MHz), δ (ppm): 137.46, 51.76, 47.03, 39.79. m/z: Found; [M+H]⁺ 511.4663, calc. for [M+H]⁺ 511.4667.

### 4. Reaction of 1,2,3,4,5,6-hexakis(bromomethyl)benzene (Ph-6-CH₂Br) with propane-,3-diamine (PD)

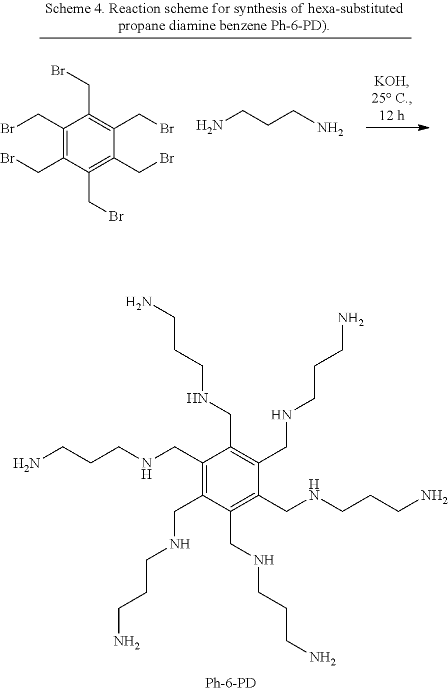 [SMILES: BrCc1c(CBr)c(CBr)c(CBr)c(CBr)c1CBr ; NCCCN ; NCCCNCc1c(CNCCCN)c(CNCCCN)c(CNCCCN)c(CNCCCN)c1CNCCCN]

Synthesis of Ph-6-PD: In a round bottom flask 1,2,3,4,5,6-hexakis(bromomethyl)benzene (1.5 g, 2.36 mmol), excess propane-1,3-diamine (10.5 g, 141.59 mmol) and KOH (0.82 g, 14.63 mmol) were added under argon atmosphere. The reaction mixture was stirred overnight at room temperature. Excess propane-1,3-diamine was removed under reduced pressure (rotary evaporation). Subsequently, the resulting colorless viscous product was dissolved in methanol (10 mL). The inorganic salt (KBr) was precipitated by addition of diethyl ether and separated by filtration. The filtrate was evaporated to afford a highly viscous yellow liquid (1.2 g, 85% yield). ¹H NMR, (D₂O, 700 MHz), δ (ppm): 3.78 (12H, s), 2.77 (12H, t), 2.67 (12H, t), 1.69 (12H, q). ¹³CNMR, (D₂O, 175 MHz), δ (ppm): 137.42, 47.22, 46.90, 38.76, 31.38. m/z: found; [M+H]⁺ 595.5602, calc. for [M+H]⁺ 595.5606.

### Synthesis of Solid Supports

### 5. Synthesis of Mesoporous SBA-15

Pluronic P-123 block copolymer ((EO)₂₀(PO)₇₀(EO)₂₀) (12 g) was dissolved in deionized water (318 mL). HCl solution (60 mL, 21.1 M) was added to the mixture and vigorously stirred for 3 h until a clear solution was formed. Tetraethyl orthosilicate (TEOS, 23.3 g) was dropwise added to the reaction mixture under constant stirring. After TEOS dropwise addition, the temperature was raised to 40° C. and held for 24 h. During this time a white precipitate has formed. Then the stirring was stopped and the temperature was raised to 100° C. for additional 20 h. Afterwards the reaction was quenched with 400 mL deionized water and the precipitate was filtered and washed copiously with deionized water. The filtered precipitate was dried for 12 h in an oven at 75° C. under ambient air conditions. The dried sample was then calcined according to the following temperature program: heat to 200° C. at 1.2° C. min⁻¹, hold at 200° C. for 1 h, heat to 550° C. at 1.2° C. min⁻¹, hold at 550° C. for 12 h, uncontrolled cooling to room temperature. The resulting white powder was stored in a glass jar at ambient conditions.

### 6. General Preparatory Procedure for Ph-XX—YY/SBA-15 Sorbents

SBA-15 was loaded with Ph-XX—YY by wet impregnation. Before use SBA-15 was dried overnight under vacuum (<20 mTorr) at 110° C. to remove trace water. Then for each composite formulation SBA-15 (200 mg) and methanol (50 mL) was mixed into a round bottom flask and stirred for 2 h. Separately, the desired amount of Ph-XX—YY was dissolved in methanol 10 mL and stirred for 10-15 min. This solution was added to SBA-15/methanol mixture and stirred overnight at room temperature. The solvent was removed under reduced pressure (rotary evaporation). The resulting powder was dried overnight under vacuum (<20 mTorr) at 50° C. All prepared composite powders were stored in glass vials at ambient conditions.

### 7. General Preparatory Procedure for Ph-XX—YY/Porous Carbon and Ph-XX—YY/γ-Al₂O₃ Sorbents

The preparation of these composites followed the same steps, conditions and amounts of materials as described above for Ph-XX—YY/SBA-15.

### Results and Discussions

1. Composites of Ph-XX—YY and mesoporous silica (SBA-15). Tri and hexa-amine substituted benzene molecules were synthesized by one-step reaction between commercially available analogous bromomethyl-substituted benzene and excess diamines (Table 1). Ph-3-ED was prepared by nucleophilic substitution between excess ethylenediamine and 1,3,5-tris(bromomethyl)benzene (Scheme 1). Dry THF solution of 1,3,5-tris(bromomethyl)benzene was added dropwise to excess ethylenediamine. Excess and slow addition is required to prevent oligomerization/polymerization. The resulting viscous oil was dissolved in KOH/methanol solution. The inorganic salts were precipitated by addition of diethyl ether and removed by filtration to obtain the desired Ph-3-ED in excellent yield (>95%). A similar strategy was employed to synthesize Ph-3-PD. Nucleophilic substitution between excess propane-1,3-diamine and 1,3,5-tris(bromomethyl)benzene gave a transparent viscous liquid in a yield of 96% (Scheme 2). The ¹H NMR spectrum of Ph-3-ED showed three protons of the phenylene ring appearing as singlets at 7.21 ppm. The proton signal of the methylene group directly connected to phenyl ring appeared as singlet at 3.73 ppm. A pair of triplets centered at 2.61 and 2.71 ppm was assigned to methylene protons belonging to the alkyl chain of the diamine: one connected to the secondary amine and the other one to the primary amine, respectively. HR-MS of the Ph-3-ED evaluated the m/z ratio as 295.2607 Da (calc. 295.2605). The ¹H NMR spectrum of Ph-3-PD showed similar peaks as compared to that of Ph-3-ED except an additional peak observed at 1.65 ppm for the additional methylene protons. HR-MS of the Ph-3-PD gave an m/z ratio of 337.3078 Da (calc. 337.3074).

Hexa-amine substituted benzene molecules were synthesized using a similar method as stated above. Hexakis(bromomethyl)benzene was treated with excess of ethylenediamine or 1,3-diaminopropane to obtain the corresponding Ph-6-ED and Ph-6-PD, respectively. As expected, the ¹H NMR spectra of Ph-6-ED and Ph-6-PD did not show any phenyl protons confirming their full substitution with alkyl diamines. Methylene protons directly connected to the phenyl ring were observed at ˜3.8 ppm for Ph-6-ED and Ph-6-PD, as was similarly observed for Ph-3-YY (˜3.72 ppm). For Ph-6-ED, the methylene protons between the primary and secondary amines were observed as multiplets (2.75-2.85 ppm). In contrast, for Ph-6-PD, two triplets were observed centered at 2.67 and 2.77 ppm for the methylene protons directly connected to the primary and secondary amines. HR-MS of the Ph-6-ED showed the molecular ion at 511.4663 Da (m/z) (511.4667 calc.). The ¹H NMR spectrum of Ph-6-PD showed similar peaks as compared to the Ph-3-ED, with an additional peak at 1.69 ppm for methylene protons. HR-MS of the Ph-6-PD observed the molecular ion at 595.5602 Da (m/z) (595.5606 calc.).

The SBA-15 support was functionalized with Ph-XX—YY using a wet impregnation method. In a typical synthesis process, the desired amount of Ph-XX—YY was dissolved in methanol (10 mL). In a separate round bottom flask, the SBA-15 support material (200 mg) was added to 50 mL of methanol and stirred vigorously for 1h. Subsequently, the Ph-XX—YY solution was added to the SBA-15/methanol suspension and the final mixture was stirred overnight at room temperature. After that, the solvent was removed using rotary evaporation and dried overnight under high vacuum. The percentage loading of the polymer in the composite was varied by using equation 1.

$$\% Loading = \frac{α}{α + β} \times 100 \qquad Equation \text{:} 1$$

where α represents the mass of added Ph-XX—YY (g) and β represents the mass of SBA-15 silica support (g).

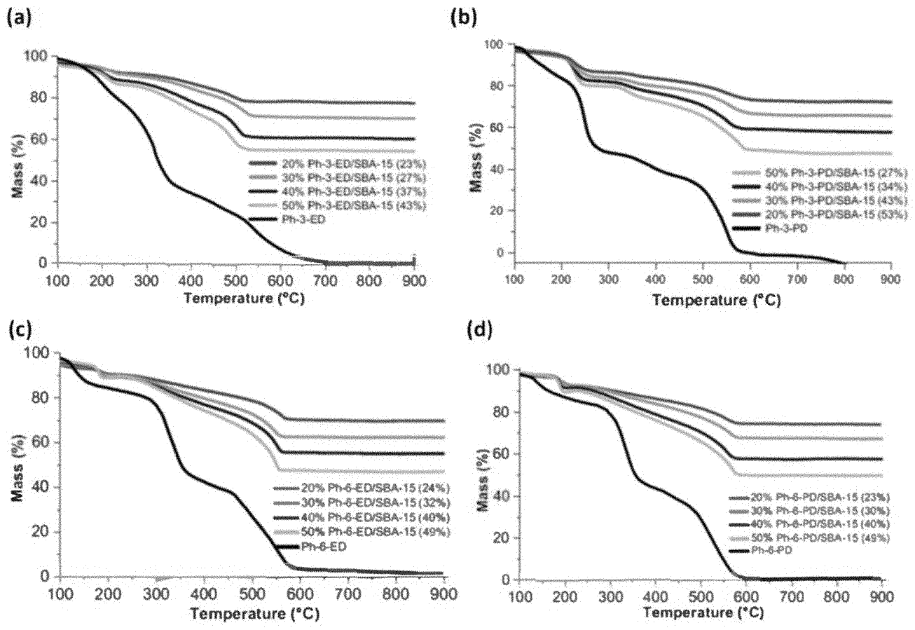

Thermogravimetric analysis (TGA) was used to evaluate the decomposition profiles of organic and inorganic constituents of the pure and Ph-X—YY/SBA-15 composites. As shown in FIG. 1, the two-step decomposition events of organic components of the samples occurred in the 120-600° C. temperature range. The first mass loss event likely associated with the volatilization of adsorbed water and/or CO₂, followed by diamine alkyl chain decomposition was observed between 120° C.-350° C. for the pure Ph-3-ED (FIG. 1a), Ph-3-PD (FIG 1b), Ph-6-ED (FIG. 1c) and Ph-6-PD (FIG. 1d). The change in mass attributed to this event was about 35-40%. In the same temperature interval, the Ph-3-PD TG trace showed an additional small signal centered around 400° C. The second major change (˜55-57%) in mass was recorded between 350° C.-600° C. Not all pure samples reached a null char after heating to 600° C.-650° C., likely due to presence of trace inorganic salts (2-5%). The decomposition profiles of the Ph-XX—YY/SBA15 composite materials followed the same patterns. The shape of the TG traces, especially that associated with the second step decomposition, suggests that impregnation in SBA-15 led to a slightly increased thermal stability when compared to pure Ph-XX—YY compounds. Overall, the evaluated mass loss percentages attributed to organic content were close to the target values used in the composite material preparation. Elemental analysis supported these findings. Table 2 summarizes the amount of nitrogen (N) found in each sample. As expected, the content of N increased with the increase in organic loading.

### See FIG. 1.

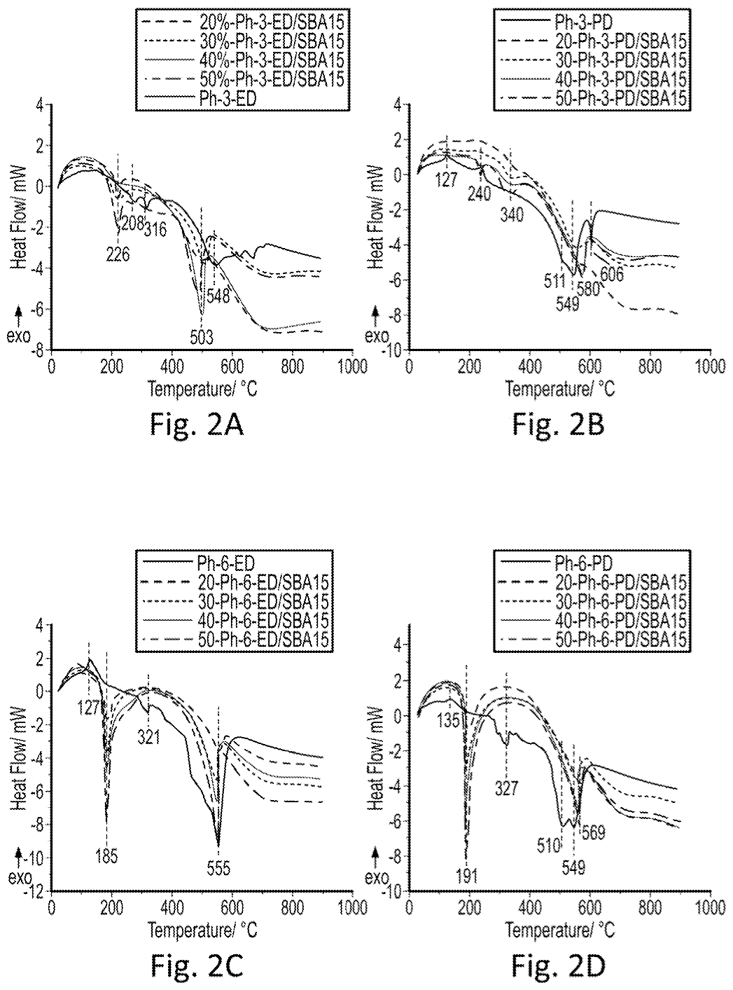

Differential scanning calorimetry (DSC) performed in tandem with TGA revealed additional information about the thermal behavior of these composite materials. FIG. 2 shows that pure Ph-XX—YY samples displayed three distinct events. A small exothermic peak was recorded between 125° C.-135° C. and was assigned to evaporation of water and/or CO₂. The second more prominent endothermic event occurred at around 300° C.-350° C. likely due to increase flexibility of alkyl chains anchored to the aromatic core. In Ph-3-PD (FIG. 2b), this event is exothermic, similar to a crystallization event. Upon further increase in temperature the aromatic cores becomes also mobile. Therefore, the third most prevalent endotherm centered at ˜500° C.-550° C. likely reflects a change in orientation of the aromatic rings. The fact that the two fragments, alkyl and aryl, have different structures and are connected through C—N bonds separates their expected onset decomposition events. At the elevated temperature (>550° C.-600° C.) most of the remaining organic content decomposed according to TG traces.

The described peaks have changed their positions in the Ph-XX—YY/SBA-15 composites. A sharp endothermic event assigned to alkyl chains gaining mobility within the SBA-15 pores was centered at about 180-225° C. The ‘fluidization’ of aromatic rings event shifted up with ˜20-30 centigrades (550° C.-580° C.). The shift in the position of the two peak ranges is likely associated with desorption of the alkyl branches and aromatic core from the support pore walls. Overall the TG/DSC data indicate that pure Ph-XX—YY and their composites with SBA-15 have a broad decomposition profile accompanied by phase and structural changes. These data suggest that confinement of Ph-XX—YY in the mesoporous support leads to good thermal stability necessary for many practical applications.

### See FIG. 2.

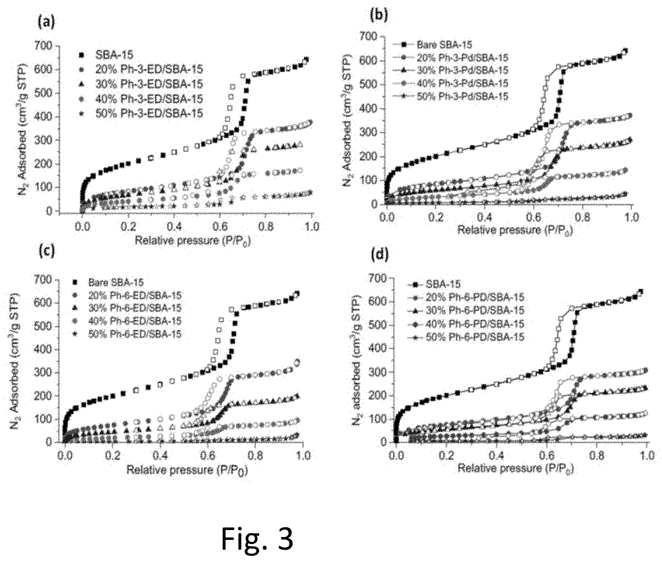

N₂ physisorption analysis was further used to determine the porosity and pore structure parameters of the support SBA-15 before and after impregnation with Ph-XX—YY molecules. As shown in FIG. 3, the adsorption isotherm of the bare SBA-15 was characterized by a sharp uptake in the region of P/P₀<0.01, corresponding to gas accumulation within the intra-wall micropores. A well-defined hysteresis loop of type H1 (IUPAC)³⁷ was recorded (0.50<P/Po<0.80) and was associated with N₂ capillary condensation and desorption in cylindrical mesopores. The shape of the loop indicates that SBA-15 consists of uniform pore networks. The pore size distribution was evaluated by the conventional BJH method. Neat SBA-15 had an average pore diameter of ˜7 nm accounting for a total pore volume of 0.96 cm³/g. The specific surface area was 700 m²/g (Table 2). After impregnation with Ph-XX—YY, the general pore structure was preserved but the average pore size decreased, reflecting the presence of the amine-molecules in the mesopores.

### See FIG. 3.

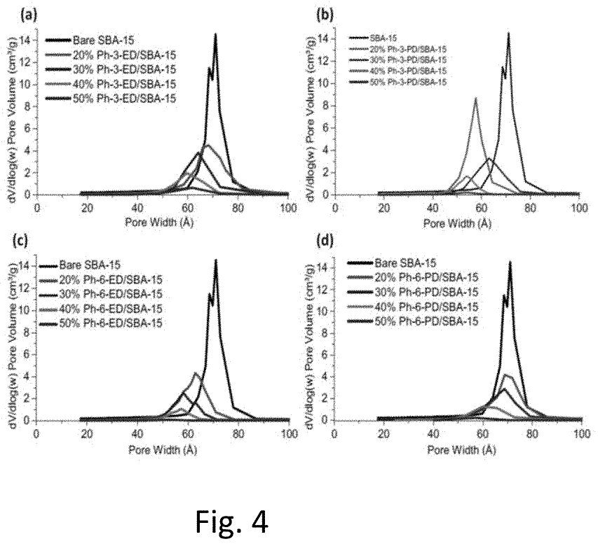

For example, as shown in Table 2, the total pore volume of 20% Ph-3 -ED/SBA-15 composite decreased from 0.96 cm³/g to 0.71 cm ³/g (26% pore filling). The reduction in the total pore volume and pore size of the SBA-15 was correlated to the increase in organic loadings. An increase of 30, 40 and 50% in organic content was reflected in a further decrease in pore volume 0.59 cm³/g, 0.41 cm³/g and 0.18 cm³/g, respectively. These values correspond to a pore filling percentage of 39, 57 and 82% respectively. The same trend was assessed by amine loadings obtained by elemental analysis (EA). The amine loadings in the samples increased from 3.3 mmole N/g at 20% loading to 11 mmole N/gSiO2 at 50% loading, coinciding with the increasing Ph-3-ED samples loading in the pores of SBA-15 (Table 2). Similar trends were observed for the other composite samples (Table 2). The calculated pore filling values for 20, 30, 40 and 50% organic loading were 20%, 40% 61% and 90% for Ph-3-PD/SBA-15, 34%, 55%, 77% and 94% for Ph-6-ED/SBA-15 and 39%, 49%, 69% and 92% for Ph-6-PD/SBA-15. The amine loadings in Ph-3-PD/SBA-15 composite increased from 3.94 to 12.45 mmol N/gSiO2 for organic loadings between 20-50%. As expected in case of Ph-6-ED and Ph-6-PD, a higher amine loading range was observed 4.54-16.7 mmol N/gSiO2 and 4.23-15.45 mmol N/gSiO2, respectively. Additionally, FIG. 4 displays the average pore size variation (˜5-7 nm) of these composite materials. Together the data presented in FIGS. 3 and 4 indicate that Ph-XX—YY molecules were distributed within cylindrical mesopores.

Another important parameter that confirmed successful impregnation of Ph-XX—YY into the SBA-15 supports was the specific surface area. As listed in Table 2, the BET surface area of bare SBA-15 was 700 m²/gSiO2. In general, variation of this parameter for the composite samples was in the 400-30 m²/g_{SiO2} range following the trend in pore filling. As expected, the slightly smaller specific surface area and slightly higher total pore filling of Ph-6-YY, as compared to the Ph-3-YY, is due to the fact that Ph-6-YY molecules are bulkier and occupy a larger volume.

### See FIG. 4.

ED/SBA-15 and (d) Ph-6-PD/SBA15 composites. Each graph contains the trace of neat SBA15.

TABLE 2
Summary of physical properties: theoretical and TGA Evaluated
Organic Content, BET Surface Area, Total Pore Volume, Pore
Filling and Amine Loading for Ph-XX-YY/SBA-15 Composites.

| | | | BET | Total | | |
|---|---|---|---|---|---|---|
| | Theoretical | | surface | pore | | Amine |
| | Organic | Organic | area | volume | Pore | Loading |
| | content | content | (m²/ | (cm³/ | filling | (mmol |
| Sample | (wt. %) | (wt. %) | g_{SiO2}) | g_{SiO2}) | (A) | N/g _{SiO2}) |
| SBA-15 | 0 | 0 | 700 | 0.96 | — | NA |
| Ph-3-ED/ | 20 | 23 | 401 | 0.71 | 26 | 3.3 |
| SBA-15 | 30 | 27 | 345 | 0.59 | 39 | 4.9 |
| | 40 | 37 | 241 | 0.41 | 57 | 7.34 |
| | 50 | 43 | 102 | 0.18 | 82 | 11 |
| Ph-3-PD/ | 20 | 27 | 375 | 0.77 | 20 | 3.94 |
| SBA-15 | 30 | 34 | 321 | 0.58 | 40 | 5.25 |
| | 40 | 43 | 204 | 0.37 | 61 | 8.22 |
| | 50 | 53 | 55 | 0.1 | 90 | 12.45 |
| Ph-6-ED/ | 20 | 24 | 367 | 0.63 | 34 | 4.54 |
| SBA-15 | 30 | 32 | 249 | 0.43 | 55 | 7.5 |
| | 40 | 40 | 127 | 0.22 | 77 | 11.44 |
| | 50 | 49 | 28 | 0.06 | 94 | 16.7 |
| Ph-6-PD/ | 20 | 23 | 357 | 0.58 | 39 | 4.23 |
| SBA-15 | 30 | 30 | 291 | 0.49 | 49 | 6.44 |
| | 40 | 40 | 163 | 0.30 | 69 | 10.02 |
| | 50 | 49 | 39 | 0.08 | 92 | 15.48 |

### 2. Composites of Ph-XX—YY and Other Porous Substrates (Mesoporous Carbon and Alumina (γ-Al₂O₃).

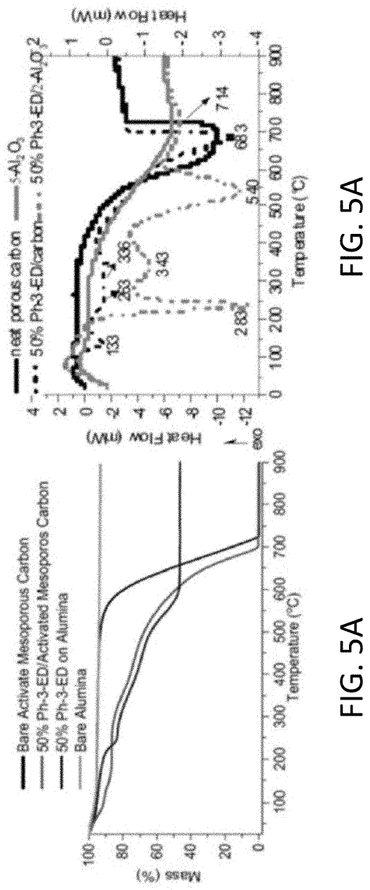

FIG. 5a shows the decomposition profile of 50% Ph-3-ED/γ-Al₂O₃ that occurred in three distinct events between 150° C.-250° C., 300° C.-450° C. and 500° C.-600° C. Similarly to Ph-3-ED/SBA-15 composites the first low temperature event was associated with the volatilization of water traces and adsorbed atmospheric CO₂. The next two decompositions were assigned to Ph-3-ED molecule. Beyond 600° C. no mass loss was observed. TGA trace of pristine porous carbon presented a single decomposition step to almost null char at around 700° C. The impregnated sample, 50% Ph-3-ED/mesoporous carbon displayed the same decomposition profile as 50% Ph-3-ED/γ-A1₂O₃ up to 600° C. Substrate burning occurred right after 650° C. and levelled out to near zero ash at 700° C. The other substrate γ-A1₂O₃ displayed remarkable temperature stability. Only approximately 7% of its mass was lost up to 900° C. Primarily, the mass was lost up 120° C. consistent with water and CO₂ loss. Both the pristine γ-A1₂O₃ substrate and the impregnated sample, 50% Ph-3-ED/γ-1₂O₃, did not burn to ash as the porous carbon and its composite material did.

The DSC traces recorded in conjunction with TGA (FIG. 5b) revealed the phase changes that accompanied the decomposition of the pure substrates and their composites. In the case of mesoporous carbon only one ample and broad endothermic event centered at 683° C. was observed. The volatilization of the water, desorption of the Ph-3-ED from the substrate walls and possibly changes in the packing occurring with the increase in temperature were recorded at 130° C., 263° C. and 336° C. The endothermic event associated with decomposition of mesoporous carbon became sharper and was centered at 683° C. DSC profile of Ph-3-ED/γ-A1₂O₃ was dominated by sharp endothermic events corresponding to decomposition of organic loading. The peaks of these events were centered at 283° C., 343° C. and 540° C. An additional small but broad endothermic signal was detected at 714° C. Likely this peak was associated with γ-A1₂O₃ phase changes occurring at elevated temperature.

### See FIG. 5.

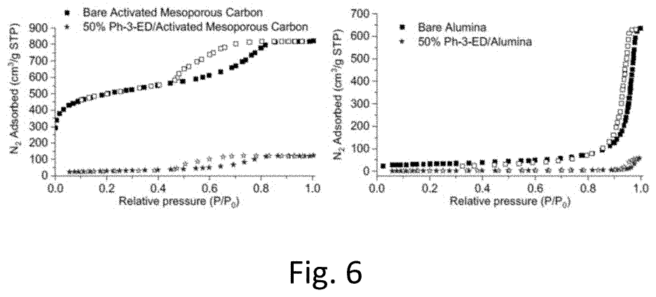

N₂ physisorption measurements were further performed to determine the pore characteristics of the two substrates and their composite materials, as shown in FIG. 6 and Table 3. Porous carbon had specific surface area (BET) of 1546 m²/g accounting for 1.26 cm³/g pore volume (FIG. 6a) while γ-A1₂O₃ had a lower BET of 113 m²/g reflecting 0.99 cm³/g pore volume (FIG. 6b) (Table 3). After impregnation with 50% Ph-3-ED, the BET surface area of Ph-3-ED/mesoporous carbon composite was significantly lower than that of the pure substrate, 99 m²/g (FIG. 6a). The pore volume followed the same trend and decreased to 0.18 cm³/g. This value indicated that 86% of the initial substrate pore volume was filled by amine sorbent. Similarly, the 50% Ph-3-ED/γ-A1₂O₃ composite had lower BET surface area (15 m²/g) and total pore volume (0.10 cm³/g) as compare to pristine γ-A1₂O₃ (FIG. 6b and Table 3). The latter value accounted for a 90% filling of the substrate pores.

### See FIG. 6.

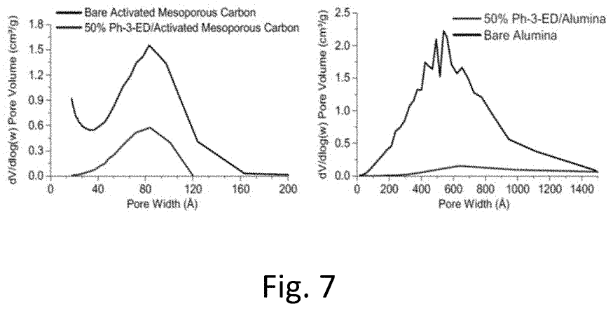

### See FIG. 7.

TABLE 3
Summary of physical properties: theoretical and TGA Evaluated
Organic Content, BET Surface Area, Total Pore Volume, Pore
Filling and Amine Loading for Ph-3-ED/mesoporous carbon and
Ph-3-ED/γ-A1₂O₃ Composites.

| | | | | Total | | Amine |
|---|---|---|---|---|---|---|
| | Theoretical | | BET | pore | | Loading |
| | Organic | Organic | surface | volume | Pore | (mmol |
| | content | content | area (m²/ | (cm³/ | filling | N/ |
| Sample | (wt. %) | (wt. %) | g_{substrate}) | g_{substrate}) | (%) | g_{substrate}) |
| Bare | 0 | 0 | 1546 | 1.26 | 0 | NA |
| Activated | | | | | | |
| mesoporous | | | | | | |
| carbon | | | | | | |
| (AMC) | | | | | | |
| 50% Ph-3- | 50 | 44 | 99 | 0.18 | 86 | 12.06 |
| ED/AMC | | | | | | |
| Bare γ- | 0 | 0 | 115 | 0.99 | 0 | NA |
| A1₂O₃ | | | | | | |
| 50% Ph-3- | 50 | 48 | 15 | 0.10 | 90 | 14.06 |
| ED/γ-A1₂O₃ | | | | | | |

### Conclusions

Three and six alkyl diamine-substituted aryl cores (Ph-XX—YY) were prepared by nucleophilic substitution from the corresponding tri and hexa bromomethyl substituted benzene with excess ethylenediamine and 1,3-diaminopropane. The one-pot synthesis of these amine-rich molecules was confirmed by ¹H, ¹³C NMR and high resolution mass spectroscopy (HR-MS). All products were obtained in high yields. Subsequently, these molecules were loaded by solution impregnation into mesoporous solid substrates (SBA-15, mesoporous carbon and γ-Al₂O₃) at various concentrations. The organic loading of the composite materials was confirmed by TGA and elemental analysis and was comparable to the theoretical value. Both DSC and TGA analyses performed on these mixtures revealed good thermal stability spanning from 180° C. to 560° C.

The porosity and pore structure of the Ph-XX—YY/solid substrate composites were assessed by N₂ physisorption measurements. The presence of the Ph-XX—YY molecules in the support pores was confirmed by a decrease in both the average pore size and surface area of the composite materials. The highest pore filling achieved for 50% organic loading was 94% for Ph-6-ED/SBA-15. This value was slightly higher when compared to Ph-3-ED/SBA-15, Ph-3-ED/mesoporous carbon and Ph-3-ED/γ-Al₂O₃ homologues. Different values were also evaluated for SBA-15-based composites containing Ph-3-ED vs. those containing Ph-3-PD. These data indicated that the molecular structure of the Ph-XX—YY was tightly associated with the pore characteristics of the composites. Importantly, results obtained from TGA and N₂ physisorption measurements showed that no Ph-XX—YY loss occurred during the preparation of the adsorbent and suggested that almost all Ph-XX—YY was introduced into the pores.

The ability to prepare thermally stable alky-aryl amine rich small molecules by a scalable one-pot approach offers great potential in using them in multiple applications. They are appealing candidates for integration as adsorbents in gas separation, direct air CO₂ capture and H₂S capture technologies.

FIG. 1: TGA curves for (a) Ph-3-ED/SBA-15, (b) Ph-3-PD/SBA15, (c) Ph-6-ED/SBA-15 and Ph-6-PD/SBA-15 composites. Each graph contains the trace of pure Ph-X—YY molecule.

FIG. 2. Differential scanning calorimetry profiles of (a) Ph-3-ED/SBA-15, (b) Ph-3-PD/SBA-15, (c) Ph-6-ED/SBA-15 and (d) Ph-6-PD/SBA-15 composites. Each graph contains the trace of pure Ph-XX—YY.

FIG. 3: Nitrogen physisorption isotherms for (a) Ph-3-ED/SBA-15, (b) Ph-3-PD/SBA-15, (c) Ph-6-ED/SBA-15, (d) Ph-6-PD/SBA-15 composites of various organic loadings (0-50%) recorded at 77K.

FIG. 4. Pore size distribution for (a) Ph-3-ED/SBA-15, (b) Ph-3-PD/SBA-15, (c) Ph-6-ED/SBA-15 and (d) Ph-6-PD/SBA15 composites. Each graph contains the trace of neat SBA15.

FIG. 5. (a) TGA and (b) DSC traces of Ph-XX—YY/mesoporous carbon and Ph-XX—YY/γ-A1₂O₃

FIG. 6. Nitrogen physisorption isotherms for (a) Ph-3-ED/mesoporous carbon and (b) Ph-3-ED/γ-A1₂O₃ composites of 0% and 50% organic loading recorded at 77K.

FIG. 7. Pore size distribution for (a) 50% Ph-3-ED/CNP and (b) 50% Ph-3-PD/γ-A1₂O₃ composites. Each graph also contains the trace of the neat substrate CNP and γ-A1₂O₃.

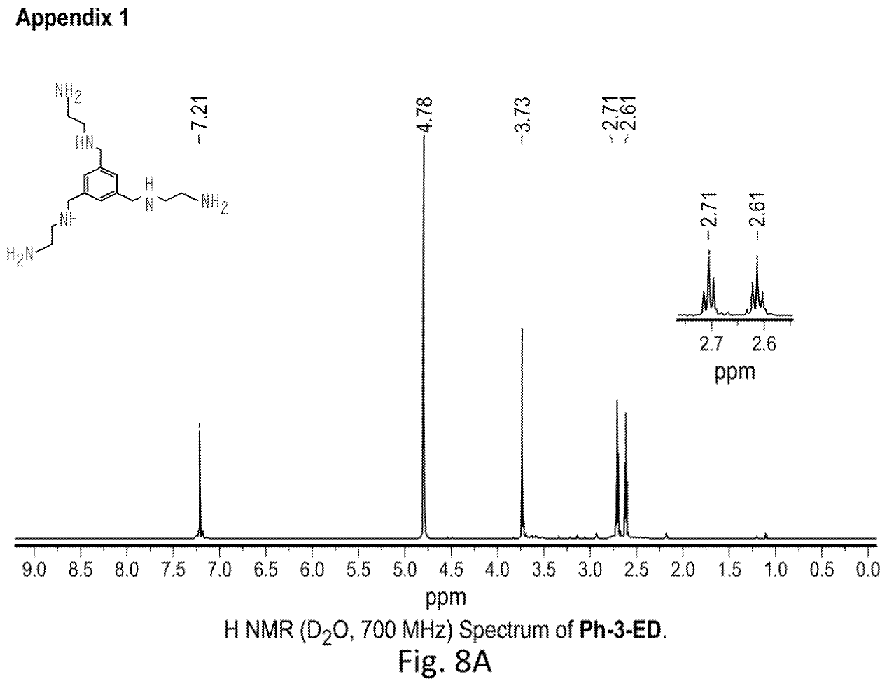

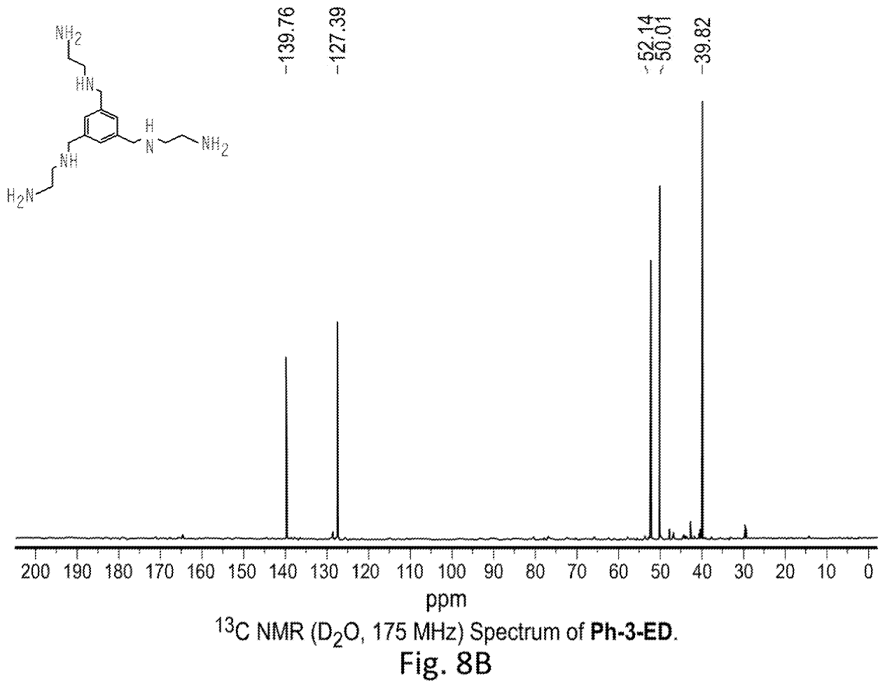

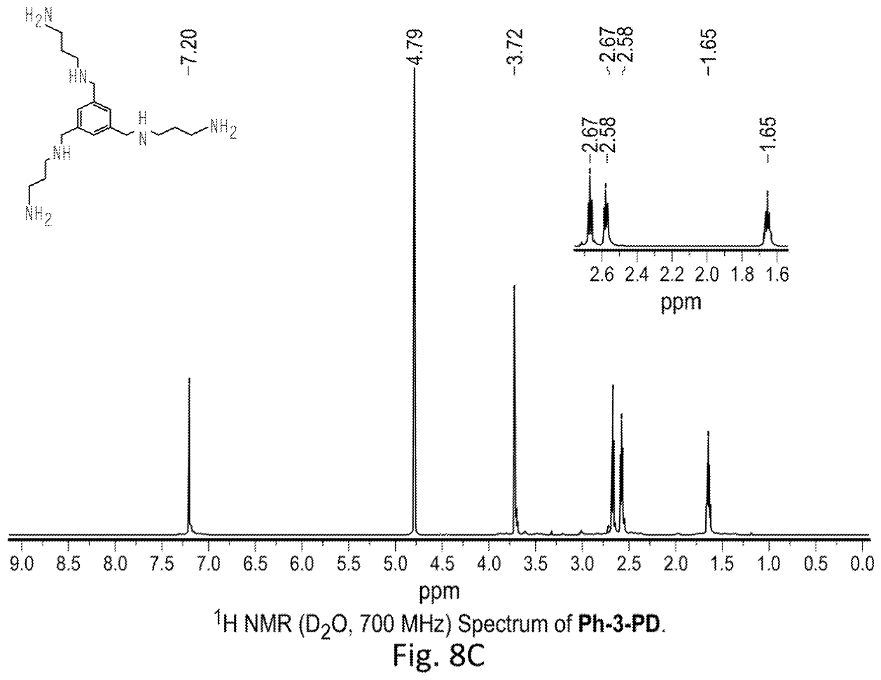

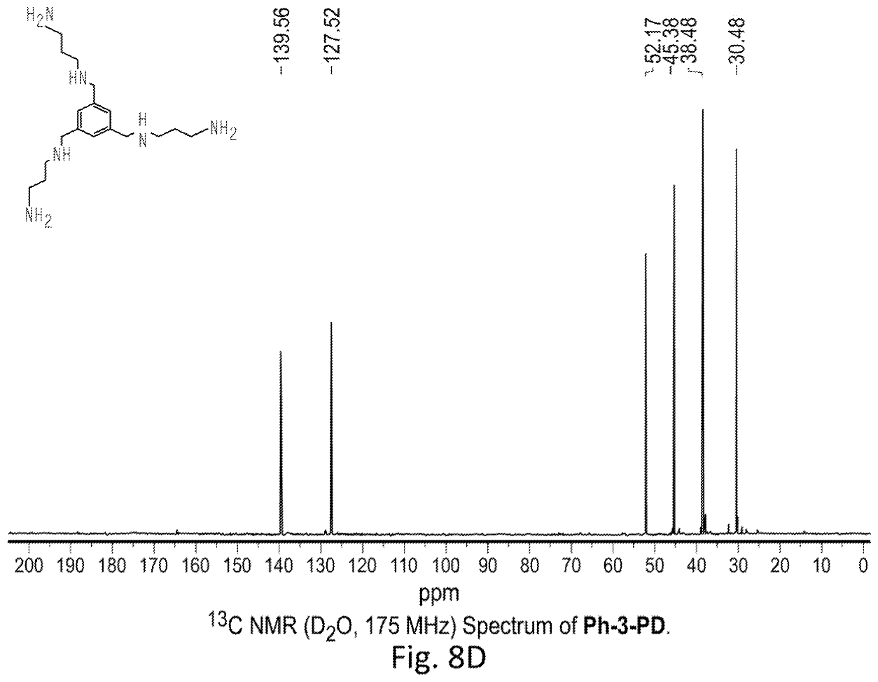

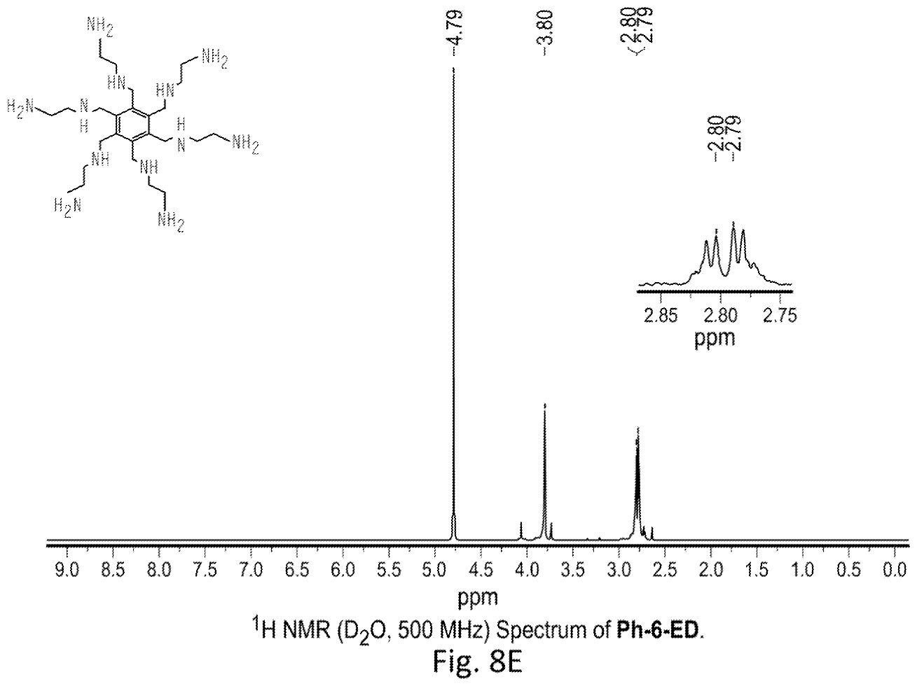

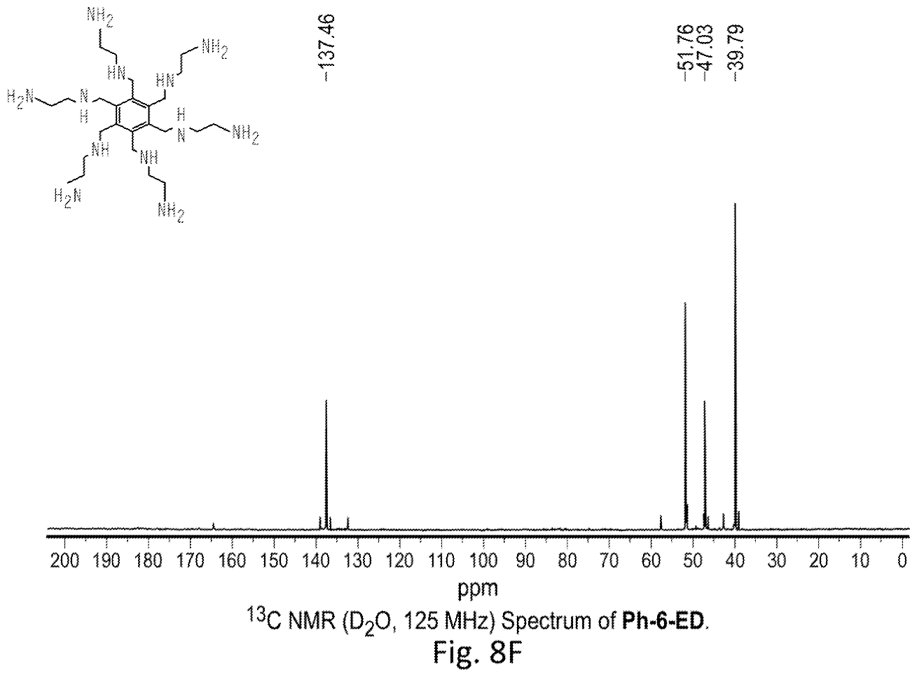

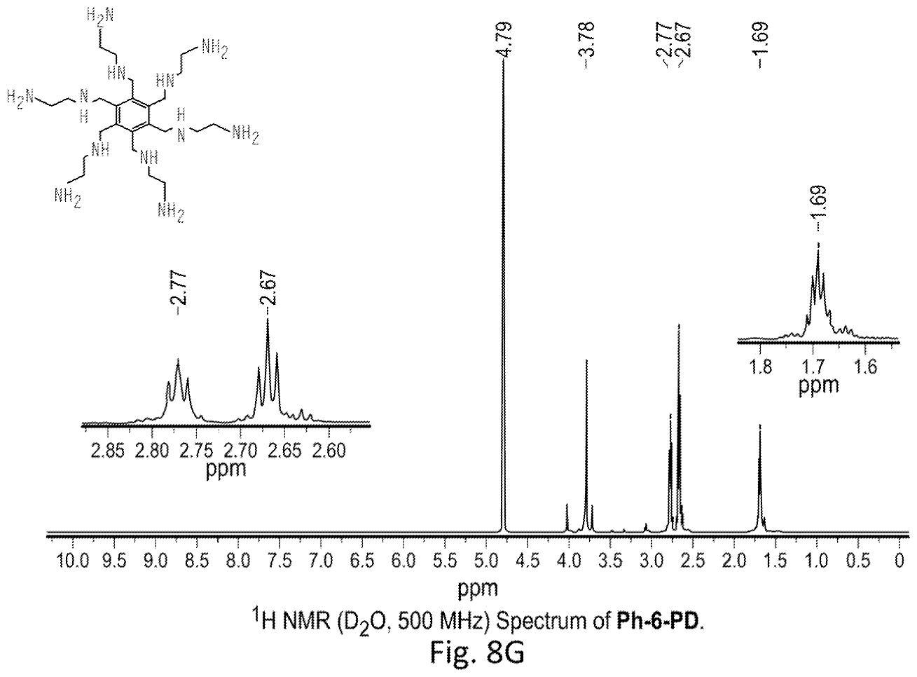

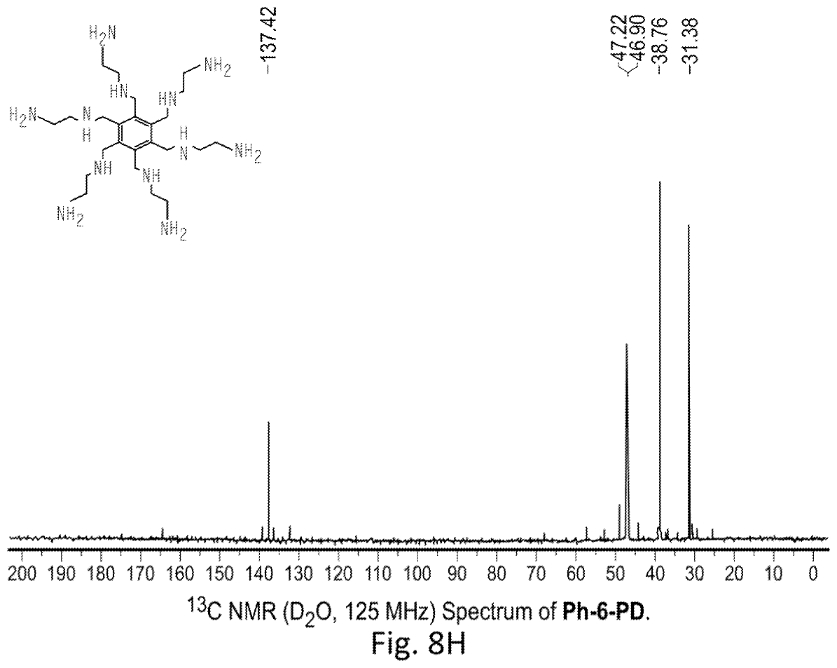

FIGS. 8A-8H illustrates NMR images of various compounds.

### REFERENCES

1. Sun, L.-B.; Kang, Y.-H.; Shi, Y.-Q.; Jiang, Y.; Liu, X.-Q., Highly Selective Capture of the Greenhouse Gas CO2 in Polymers. ACS Sustain. Chem. & Eng. 2015, 3, 3077-3085.

2. Sun, L.-B.; Li, A.-G.; Liu, X.-D.; Liu, X.-Q.; Feng, D.; Lu, W.; Yuan, D.; Zhou, H.-C., Facile fabrication of cost-effective porous polymer networks for highly selective CO2 capture. J. Mater. Chem. A 2015, 3, 3252-3256.

3. Looper, R.; Williams, D.; Jeyapalina, S.; Haussener, T.; Sebahar, P. R.; Reddy Hariprasada, R. K. Compositions and Methods Comprising a Biocidal Polyamine. US 20150038512 A1, 2016.

4. Looper, R.; Williams, D.; Jeyapalina, S.; Haussener, T.; Sebahar, P. R.; Reddy Hariprasada, R. K. Methods of use for Compositions Comprising a Biocidal Polyamine. US 20140350017 A1, 2014.

5. Sawicki, M.; Lecerclé, D.; Grillon, G.; Le Gall, B.; Sérandour, A.-L.; Poncy, J.-L.; Bailly, T.; Burgada, R.; Lecouvey, M.; Challeix, V.; Leydier, A.; Pellet-Rostaing, S.; Ansoborlo, E.; Taran, F., Bisphosphonate sequestering agents. Synthesis and preliminary evaluation for in vitro and in vivo uranium(VI) chelation. Euro. J. Med. Chem. 2008, 43, 2768-2777.

6. Guo, Y.-H.; Ge, Q.-C.; Lin, H.; Lin, H.-K.; Zhu, S.-R., Thermodynamic studies on supramolecular interactions of metal ions with nucleotides/tripods ligands. Polyhedron 2002, 21, 1005-1015.

7. Guo, Y.; Ge, Q.; Lin, H.; Zhu, S.; Lin, H., The role of copper(II) ion in regulating H-bonding and coulombic interactions in copper(II)/tripod/polyphosphate systems. Transition Metal Chemistry 2003, 28, 609-615.

8. Guo, Y.-H.; Ge, Q.-C.; Lin, H.-K.; Zhu, S.-R.; Lin, H., Stability studies on 1:1 or 1:3 stoichiometry complexes of hexaza tripods with zinc(II), copper(II), nickel(II) and cobalt(II). Transition Metal Chemistry 2004, 29, 42-45.

9. Still Clark, W.; Li, G.; Wennemers, H. Synthetic receptors. US 6797522 B1, 2004.

10. Seipp, C. A.; Williams, N. J.; Kidder, M. K.; Custelcean, R., CO2 Capture from Ambient Air by Crystallization with a Guanidine Sorbent. Angew. Chem. Int. Edit. 2017, 56, 1042-1045.

11. Nagata, M.; Kiyotsukuri, T.; Moriya, T.; Tsutsumi, N.; Sakai, W., Novel regular network polyimide films from mellitic acid and aliphatic and aromatic diamines or diisocyanates. Polymer 1995, 36, 2657-2662.

12. Kiyotsukuri, T.; Murata, M.; Sugimoto, M.; Tsutsumi, N., Regular network polyamide and copolyamide films from trimesic acid and aliphatic diamines. Polym. Int. 1994, 34, 173-179.

13. Kiyotsukuri, T.; Tsutsumi, N.; Okada, H.; Nagata, M., Novel regular network wholly aromatic polyester and polyamide films. Polymer 1992, 33, 4990-4996.

14. Jikei, M.; Chon, S.-H.; Kakimoto, M.-a.; Kawauchi, S.; Imase, T.; Watanebe, J., Synthesis of Hyperbranched Aromatic Polyamide from Aromatic Diamines and Trimesic Acid. Macromolecules 1999, 32, 2061-2064.

15. Edwards, M. W.; Robinson, M. I. Diamine Salts of Pyromellitic Acid Diester. U.S. Pat. No. 2,880,230, 1959.

16. Yu, S.; Lee, W.; Seo, B.; Lim, C.-S., Synthesis of Benzene Tetracarboxamide Polyamine and Its Effect on Epoxy Resin Properties. Polymers 2018, 10, 782.

17. Kumar, D., Condensation polymerization of pyromellitic dianhydride with aromatic diamine in aprotic solvent: A reaction mechanism. Journal of Polymer Science: Polym. Chem. Edition 1981, 19, 795-805.

18. Sanz-Perez, E. S.; Murdock, C. R.; Didas, S. A.; Jones, C. W., Direct Capture of CO2 from Ambient Air. Chem. Rev. 2016, 116, 11840-11876.

19. Li, K.; Jiang, J.; Tian, S.; Yan, F.; Chen, X., Polyethyleneimine-nano silica composites: a low-cost and promising adsorbent for CO2 capture. J. Mater. Chem. A 2015, 3, 2166-2175.

20. Hicks, J. C.; Drese, J. H.; Fauth, D. J.; Gray, M. L.; Qi, G.; Jones, C. W., Designing Adsorbents for CO2 Capture from Flue Gas-Hyperbranched Aminosilicas Capable of Capturing CO2 Reversibly. J. Am. Chem. Soc. 2008, 130, 2902-2903.

21. Estevez, L.; Barpaga, D.; Zheng, J.; Sabale, S.; Patel, R. L.; Zhang, J.-G.; McGrail, B. P.; Motkuri, R. K., Hierarchically Porous Carbon Materials for CO2 Capture: The Role of Pore Structure. Ind. Eng. Chem. Res. 2018, 57, 1262-1268.

22. Kong, W.; Liu, J., Ordered mesoporous carbon with enhanced porosity to support organic amines: efficient nanocomposites for the selective capture of CO2. New J. Chem. 2019, 43, 6040-6047.

23. Xu, X.; Song, C.; Andresen, J. M.; Miller, B. G.; Scaroni, A. W., Novel Polyethylenimine-Modified Mesoporous Molecular Sieve of MCM-41 Type as High-Capacity Adsorbent for CO2 Capture. Energy & Fuels 2002, 16, 1463-1469.

24. Pang, S. H.; Lively, R. P.; Jones, C. W., Oxidatively-Stable Linear Poly(propylenimine)-Containing Adsorbents for CO2 Capture from Ultradilute Streams. ChemSusChem 2018, 11, 2628-2637.

25. Sakwa-Novak, M. A.; Tan, S.; Jones, C. W., Role of Additives in Composite PEI/Oxide CO2 Adsorbents: Enhancement in the Amine Efficiency of Supported PEI by PEG in CO2 Capture from Simulated Ambient Air. ACS Appl. Mater. Interfaces 2015, 7, 24748-24759.

26. Bali, S.; Leisen, J.; Foo, G. S.; Sievers, C.; Jones, C. W., Aminosilanes Grafted to Basic Alumina as CO2 Adsorbents—Role of Grafting Conditions on CO2 Adsorption Properties. ChemSusChem 2014, 7, 3145-3156.

27. Sakwa-Novak, M. A.; Yoo, C.-J.; Tan, S.; Rashidi, F.; Jones, C. W., Poly(ethylenimine)-Functionalized Monolithic Alumina Honeycomb Adsorbents for CO2 Capture from Air. ChemSusChem 2016, 9, 1859-1868.

28. Wang, Y.; Du, T.; Qiu, Z.; Song, Y.; Che, S.; Fang, X., CO2 adsorption on polyethylenimine-modified ZSM-5 zeolite synthesized from rice husk ash. Mater. Chem. Phy. 2018, 207, 105-113.

29. Liu, Y.; Sajjadi, B.; Chen, W.-Y.; Chatterjee, R., Ultrasound-assisted amine functionalized graphene oxide for enhanced CO2 adsorption. Fuel 2019, 247, 10-18.

30. Deng, J.; Liu, Z.; Du, Z.; Zou, W.; Zhang, C., Fabrication of PEI-grafted porous polymer foam for CO2 capture. J. Appl. Polym. Sci. 2019, 136, 47844.

31. Peng, A.-Z.; Qi, S.-C.; Liu, X.; Xue, D.-M.; Peng, S.-S.; Yu, G.-X.; Liu, X.-Q.; Sun, L.-B., Fabrication of N-doped porous carbons for enhanced CO2 capture: Rational design of an ammoniated polymer precursor. Chem. Eng. J. 2019, 369, 170-179.

32. Li, J.; Michalkiewicz, B.; Min, J.; Ma, C.; Chen, X.; Gong, J.; Mijowska, E.; Tang, T., Selective preparation of biomass-derived porous carbon with controllable pore sizes toward highly efficient CO2 capture. Chem. Eng. J. 2019, 360, 250-259.

33. Azmi, A. A.; Aziz, M. A. A., Mesoporous adsorbent for CO2 capture application under mild condition: A review. J. Environ. Chem. Eng. 2019, 7, 103022.

34. Hou, S.-L.; Dong, J.; Jiang, X.-L.; Jiao, Z.-H.; Zhao, B., A Noble-Metal-Free Metal-Organic Framework (MOF) Catalyst for the Highly Efficient Conversion of CO2 with Propargylic Alcohols. Angew. Chem. Int. Edit. 2019, 58, 577-581.

35. Liu, L.; Ke, C.-C.; Ma, T.-Y.; Zhu, Y.-P., When Carbon Meets CO2: Functional Carbon Nanostructures for CO2 Utilization. J. Nanosci. Nanotechnol. 2019, 19, 3148-3161.

36. Hussain, A.; Farrukh, S.; Hussain, A.; Ayoub, M., Carbon capture from natural gas using multi-walled CNTs based mixed matrix membranes. Environ. Technol. 2019, 40, 843-854.

37. Ravikovitch, P. I.; Neimark, A. V., Characterization of Micro- and Mesoporosity in SBA-15 Materials from Adsorption Data by the NLDFT Method. J. Phys. Chem. B 2001, 105, 6817-6823.

### Brief Technical Description

Alkyl-aryl amine-rich small molecules (Ph-XX—YY) were prepared by nucleophilic substitution from tri- and hexa-bromine-substituted aromatic cores with various aliphatic diamines. The resulting products were subsequently introduced by solution impregnation into solid mesoporous supports. Various types of Ph-XX—YY molecules filled the support's pores up to ˜90% and displayed good thermal stability.

Amine molecules or polymers supported on/in solid supports are known materials with diverse applications. Here we synthesize a family of amine molecules based on a phenyl core with alkyl amine branches and incorporate these materials on/in solid supports. While some related amine molecules with an aromatic core are known (see prior art), many molecules reported here are new. While solid-supported forms of other amine molecules and polymers are known, these compositions are new, with no reports of supported versions of these amine molecules known. The alky-aryl amine rich small molecules are thermally stable. They are made in the one-step process, with mild reaction conditions and chemicals that do not prose safety concerns. Impregnation of these molecules into mesoporous substrates provides them higher thermal stability when compared to linear alkyl amines.

The present invention discloses the preparation of amine-rich small molecules (Ph-XX—YY) of various compositions. Nucleophilic substitution was employed to react tri- and hexa-bromine-substituted aromatic cores with excess ethylenediamine and propane-1,3-diamine. The excess diamine prevents oligomerization and favored high yields of products. Synthesis of alky-aryl amines was confirmed by 1H, 13C NMR and high-resolution mass spectrometry (HR-MS). Also escribed composite materials made of various organic loadings of Ph-XX—YY molecules combined with various mesoporous support materials; first with SBA-15 mesoporous silica a prototypical example, through solution impregnation. In addition, porous supports such as alumina, carbon, titania, porous polymers, and MOFs can also be used. The wet impregnation approach yielded composite materials, such as Ph-XX—YY/SBA-15. Thermogravimetric analysis (TGA) and differential scanning calorimetry (DSC) were used to investigate the thermal behavior of the pure Ph-XX—YY and composite materials. It was found that encapsulation in the SBA-15 pores shields Ph-XX—YY molecules, slowing their thermal degradation. N2 physisorption analysis was used to assess the porosity of the composite materials and determine the pore structure parameters of the Ph-XX—YY/SBA-15 composites. The specific Bet surface area decreased with the increase in Ph-XX—YY loadings, a trend also followed by the total pore volume and average pore size. These results confirmed the presence of Ph-XX—YY within the cylindrical pores of SBA-15. Together the data indicate that, due to their good thermal stability and uniform distribution within mesoporous solid supports, Ph-XX—YY/solid mesoporous support (e.g., silica, y-A1203, porous carbon) composites are appealing platforms for various applications, in particular integration into direct air CO2 capture (DAC) or other CO2 capture technologies as well as H2S capture.

What are the commercial applications for the invention?

The capture of acid gasses from gas mixtures, including (CO2, SO2, NO, NO2, H2S, etc.). While Global Thermostat has an interest primarily in CO2, we have also shown the materials to be effective for H2S capture, which could open the field of licensees substantially. A separate IP will be filed on H2S capture application with different inventors.

What are the advantages of the invention over present technologies?

The alkyl-aryl small molecules contain a high amount of amine groups and due to the aryl core, have better thermal stability than regular linear alkyl amines. Impregnation of these molecules into mesoporous substrates further increases their thermal stability.

1. (canceled)

2. Composite materials of Ph-XX—YY amines impregnated into porous supports.

3. The same as claim 2 with the support being mesoporous silica.

4. The same as claim 3 with the silica being SBA15.

5. The same as claim 2 with the support being porous alumina.

6. The same as claim 2 with the support being porous carbon. (Original) The same as claim 2 with the support being porous polymer.

8. The same as claim 2 with the support being porous metal organic frameworks.

9. The same as claim 2 with the Ph-XX—YY being Ph-3-ED (N1,N1′,N1″-(benzene-1,3,5-triyltris(methylene))tris(ethane-1,2-diamine)).

10. The same as claim 2 with the Ph-XX—YY being Ph-3-PD (N1,N1′,N1″-(benzene-1,3,5-triyltris(methylene))tris(propane-1,3-diamine)).

11. The same as claim 2 with the Ph-XX—YY being Ph-6-PD (N1,N1′,N1″,N1′″,N1″″,N1′″″-(benzene-1,2,3,4,5,6-hexaylhexakis(methylene))hexakis(propane-1,3-diamine).

12. The same as claim 2 with the Ph-XX—YY being Ph-6-ED (N1,N1′,N1″,N1′″,N1″″,N1′″″-(benzene-1,2,3,4,5,6-hexaylhexakis(methylene))hexakis(ethane-1,2-diamine)).

13. The same as claim 2 with the Ph-XX—YY being Ph-4-ED (N1,N1′,N1″,N1′″-(benzene-1,2,4,5-tetrayltetrakis(methylene))tetrakis(ethane-1,2-diamine)).

14. The same as claim 2 with the Ph-XX—YY being Ph-4-PD (N1,N1′,N1″,N1′″-(benzene-1,2,4,5-tetrayltetrakis(methylene))tetrakis(propane-1,3-diamine)).

15. The same as claim 2 with the Ph-XX—YY being Ph-3-TEPA (N1,N1′,N1″-(benzene-1,3,5-triyltris(methylene))tris(N2-(2-((2-((2-aminoethyl)amino)ethyl)amino)ethyl)ethane-1,2-diamine)).

16. The same as claim 2 with the Ph-XX—YY being Ph-3-PEI (N1,N1′,N1″-(benzene-1,3,5-triyltris(methylene))tris(polyethylenimine)).

17. The same as claim 2 with the Ph-XX—YY being Ph-2-ED (N1,N1′-(1,4-phenylenebis(methylene))bis(ethane-1,2-diamine)).

18. The same as claim 2 with the Ph-XX—YY being Ph-2-PD(N1,N1′-(1,4-phenylenebis(methylene))bis(propane-1,3-diamine)).

19-24. (canceled)
